(19) United States
(12) Patent Application Publication
(10) Pub. No.: US 2025/0152020 A1
(43) Pub. Date: May 15, 2025

(54) SYSTEM AND METHOD FOR OPTIMIZING THE FOOT PARTIAL WEIGHT LOADING FEEDBACK AND DEEP VEIN THROMBOSIS PREVENTION

(71) Applicant: Orthonix Inc., Orlando, FL (US)

(72) Inventors: Amir Reza KACHOOEI, Orlando, FL (US); Ashkan Sedigh, Orlando, FL (US)

(21) Appl. No.: 18/944,293

(22) Filed: Nov. 12, 2024

Related U.S. Application Data
(60) Provisional application No. 63/598,112, filed on Nov. 12, 2023.

(51) Int. Cl. A61B 5/02; A61B 5/00; A61B 5/103; A61B 5/1455

(52) U.S. Cl. CPC A61B 5/02007; A61B 5/1036; A61B 5/14552; A61B 5/6807; A61B 5/7264; A61B 5/742; A61B 5/746; A61B 2560/0214; A61B 2560/0462; A61B 2562/0247

(57) ABSTRACT

A postop shoe includes a body for receiving at least a part of a patient's foot, the body has a sole, a sensor placed within the body, a near infrared spectroscopy (“NIRS”) sensor and a central processor. The sensor configured to measure pressure applied on the sole. The NIRS sensor configured for detecting oxygen saturation in a desired tissue. The central processor configured for receiving signals from the sensor and the NIRS sensor, wherein the central processor is configured to send an alert signal when the sensor measures weight exceeding a predetermined weight or the NIRS sensor detects an indicator of an evolving deep vein thrombosis (“DVT”).

### CROSS-REFERENCE TO RELATED APPLICATIONS

The present application claims the benefit of U.S. Provisional Patent Application No. 63/598,112, filed Nov. 12, 2023 and titled, “System and Method for Optimizing the Foot Partial Weight Loading Feedback and Deep Vein Thrombosis (DVT) Prevention,” the entire contents of which is incorporated herein by reference in its entirety.

### BACKGROUND OF THE INVENTION

According to Wolff's law, mechanical loading is a significant factor in bone turnover, required during bone healing, such that when stress and loading is applied to the bone, the bone remodels itself to adapt to the stress. The number of trabeculae typically correlates with the amount and duration of mechanical strain on the bone. The balance between osteoblasts and osteoclasts modulates trabeculae formation. Bone loading increases osteoblast activity resulting in new bone formation while unloading the bone grows osteoclast activity resulting in bone resorption.

Underloading a fracture or an operated bone and joint may result in a nonunion or delayed union because of diminished bone formation, while overloading during recovery may result in fracture displacement, and failure of the bone, ligament, or tendon fixation. During the recovery period after surgery, the healing process is affected by the amount of exerted force by the patient and the distribution of loading strain along the bone and joint. There is a five to ten percent (5-10%) chance of complications following surgery about the lower extremity, which may be impacted by underloading or overloading the bone.

Diabetes mellitus is one of the conditions that bring about various side effects for a patient, one of which is diabetic foot ulcers due to decreased sensation and diminished circulation. Nearly fifteen percent (15%) of diabetic patients are affected by this side effect because of neuropathy and/or vasculopathy. Diabetes can result in several symptoms such as ulcer and foot infections that sometimes result in amputation at different levels. Due to a defective nervous system, these patients cannot sense excessive forces and pressure about their foot, which can result in fractures, deformities, and recalcitrant ulcers.

Besides bone healing, soft tissue also requires immobilization and reduction in the limb's exerted load. However, complete immobilization and no weight-bearing may result in muscle atrophy, which typically lengthens a patient's rehabilitation. Following a ligament or tendon repair or reconstruction, enough weight bearing is required to avoid muscle atrophy, but excessive weight bearing brings a risk of repair failure.

A disadvantage of non-weight bearing is blood clot formation in the deep veins, thereby increasing the risk of thromboembolic events in the legs, pulmonary embolism, and death. At the same time, overloading may cause fracture displacement, joint subluxation, failure of fixation or repair, and increase the chance of bleeding.

Deep Vein Thrombosis (“DVT”) is a condition that occurs when a blood clot forms in the patient's deep veins in the lower limbs. This clot can form emboli which can block the proper blood flow to different organs including the patient's brain or lungs resulting in a life-threatening event. Weight bearing is a preventive measure against blood clot formation. In a non-weightbearing status following a fracture or surgery, preventive measures include blood-thinner medication, and pneumatic compression therapy.

Early weight bearing is preferred; however, early weight bearing is preferably weighed against treatment failure. Early weight bearing necessitates programmed weight bearing based on the limb circulation to optimize the applied weight and forces applied on the affected limb. In a pre-DVT status, it is recommended to bear weight as much as allowed to improve blood circulation and prevent blood clot formation. However, if the DVT forms, weight bearing should be restricted to avoid an emboli being thrown to the lungs or otherwise travelling through the bloodstream to block a vessel, which is a life threatening event. One way to traditionally educate patients regarding the appropriate amount of allowed for weight-bearing while healing is using a bathroom scale. For instance, an example plan for a scheduled increase in weight-bearing is twenty-five percent (25%) for the first two weeks, followed by fifty percent (50%), seventy-five percent (75%), and then one hundred percent (100%) for each following two weeks. Patients may use a bathroom scale to put pressure and weight on the limb until the load for force reaches twenty-five percent (25%) of their actual weight. By repeatedly stepping on and off the plate or scale, the patient gains a feeling for the twenty-five percent (25%) weight of their body weight and tries to continue this amount of pressure or to avoid exceeding this pressure for the next two weeks with assistance from crutches or other partial weight bearing assistance to bear the remainder of the patient's weight. This strategy for the application of weight to the healing limb is a cumbersome, inaccurate and unpredictable and is prone to human error, both by underloading and overloading the limb.

Alternatively, patients may be given certain types of post operative (“postop”) shoes, such as the post-operative shoes for use after forefoot surgery described in European Patent No. EP 0 557 409 B1, to protect the vulnerable part of the sole from external forces or pressure.

For postoperative care and fracture care, orthopedic experts may recommend that patients apply the minimum weight on the affected foot initially and gradually increase the amount of weight based on the improvement or progress of healing and the patient's tolerance.

In sports such as soccer, athletes are given specialty sneakers, spikes or shoes that can measure motion, pressure, and other technical data so that their coaches can analyze the players' performance. These sneakers, spikes or shoes are equipped with several sensors embedded inside a flexible insole and a wireless transmitter to send the players' data to the coach's smartphone, tablet, etc.

More generally, smart postop shoes can provide analytical data such as location, speed, acceleration, muscle oxygenation, pressure, etc., for various purposes. One of the main issues of this type of footwear is charge consumption and the amount of time it takes to recharge the batteries. Meanwhile, the user is unable to wear the shoes.

This section is intended to provide a background or context to the invention that is described herein. The description herein may include concepts that could be pursued but are not necessarily ones that have been previously conceived or pursued. Therefore, unless otherwise indicated herein, what is described in this Background of the Invention section is not prior art to the description and claims in this application and is not admitted as prior art by inclusion in this section.

### BRIEF SUMMARY OF THE INVENTION

The present disclosure provides, among others, solutions addressing the above-mentioned problems with the existing systems. This description provides complementary improvements that may be applied separately or in combination.

In one broad aspect, the present disclosure provides a smart postop shoe including a sensor located within the postop shoe for measuring weight bearing on the postop shoe, a first near infrared spectroscopy (“NIRS”) sensor for connecting to a first area of the user body, wherein the NIRS sensor measures a first area oxygen level and first area blood volume level, in the area and a controller receiving the weight bearing and the blood flow measurements from the NIRS sensor and providing a response upon detecting that the weight bearing is not within a defined range for a predetermined oxygen level or blood volume level.

In one preferred embodiment, the postop shoe also includes a NIRS sensor for connecting to a second area of the user body and measuring a second area oxygen level or a second area blood volume level, wherein a defined or predetermined range is based on comparison between the first area oxygen level and the second area oxygen level or the first area blood volume level and the second area blood volume level.

In some preferred embodiments, the defined range may be based on a predefined table of weight bearing for different blood volume levels. In another preferred embodiment, the defined range may be based on a predefined table of weight bearing for different oxygen levels.

In some preferred embodiments, the controller may comprise a microcontroller and a memory storing information received from the sensor and the NIRS sensors.

In some preferred embodiments, the postop shoe may further comprise a rechargeable power system.

In one example of the present disclosure, the rechargeable power system comprises a power management system, and power generators.

In some embodiments of the present disclosure, the sensor measures an amount of weight in different insole areas.

In one preferred example, the postop shoe further comprises a user interface providing information regarding the weight bearing on the shoe. In one example, the postop shoe provides an alarm when the weight bearing is not in the desired range. In some preferred embodiments, the postop shoe may include a transceiver for communicating with an end device. In one embodiment of the present disclosure, the end device is a cellphone or smartphone of a user or medical professional.

In some preferred embodiments of the present disclosure, the controller is programmable by the user to determine and trigger a threshold of the pressure applied on each pressure sensor.

In some preferred embodiments of the present disclosure, the power generators of the power management system can be piezoelectric, flexible solar panels, or any pressure-based power generator.

In some preferred embodiments of the present disclosure pressure and weight sensors could be force sensitive resistor (“FSR”) sensors, piezoelectric sensors, strain gauges, or any other pressure, force and/or weight measuring sensors.

In some preferred embodiments of the present disclosure, the postop shoe alert can be a voice or vibration that is activated when the force or weight applied to the shoe is outside of a predetermined range.

In some preferred embodiments of the present disclosure, the user interface screen specifies the area of the sole of the postop shoe on which the weight has exceeded the scheduled value.

In some preferred embodiments of the present disclosure, the processor, central processor or central processing unit performs data analysis on the raw data received from the pressure sensors or any other sensors associated with the postop shoe or of the system and stores the processed input in the data storage unit.

In some preferred embodiments of the present disclosure, the system includes NIRS sensors to detect the oxygen saturation in the desired tissue.

In some preferred embodiments of the present disclosure, the sensors and oxygen threshold level parameters are calibrated with the initial dataset or an individual healthy limb.

In some preferred embodiments of the present disclosure, the system is configured to predict and diagnose the deep vein thrombosis (“DVT”) in the individual tissue.

In some preferred embodiments of the present disclosure, the system is configured to adapt and modify the weight-bearing plan to prevent the deep vein thrombosis.

In some preferred embodiments of the present disclosure, the near infrared spectroscopy is placed at the desired location on the individual limb.

In some preferred embodiments of the present disclosure, wherein the postop shoe is configured to learn supervised/unsupervised and adapt the customized weight bearing plan based on the healthy limb or relevant clinical dataset.

In some preferred embodiments of the present disclosure, wherein the postop shoe is configured to alert the physician and patient if the data diagnose or predicts a probable deep vein thrombosis.

In some preferred embodiments of the present disclosure, wherein the postop shoe is configured to reciprocally adjust the allowed weight-bearing based on the DVT status.

In some preferred embodiments of the present disclosure, the insole is custom made or designed, reshaped and manufactured based on the patient's specific complications and weight-bearing plan.

In some preferred embodiments of the present disclosure, wherein the postop shoe is configured to accept add-on outsoles, such as a rocker bottom outsoles to create a stream of load from the hindfoot to the forefoot during a stance phase of the gait.

In some preferred embodiments of the present disclosure, wherein the postop shoe is configured to accept the add-on outsoles such as the heel only outsole to unload other areas of the foot while the hindfoot loading is adjusted during the healing process.

In one other broad aspect, the present disclosure provides a method for supervising weight bearing of a user using a postop shoe. The method comprises measuring weight bearing of the postop shoe using a sensor, measuring a first area oxygen level or a first area blood volume level in a body area of the user using the NIRS sensor, defining a range for the weight bearing using the first area oxygen level or the first area blood volume level and providing a response upon detecting that the weight bearing is not within the defined range.

In some examples, the defining the range for the weight bearing using the first area oxygen level or the first area blood volume level comprises comparing the first area oxygen level to a second area oxygen level.

In some examples, the defining the range for the weight bearing using the first area oxygen level or the first area blood volume level comprises comparing the first area blood flow level to a second area blood flow level.

In some examples, the defining the range for the weight bearing using the first area oxygen level or the first area blood volume level includes using a predefined weight bearing chart for different oxygen levels.

In some examples, the defining the range for the weight bearing using the first area oxygen level or the first area blood volume level includes using a predefined weight bearing chart for different blood flow levels.

In one broad aspect, the present disclosure provides a shoe comprising a body for receiving at least a part of a patient's foot, wherein the body includes an insole, a sensor is placed within the body to measure pressure applied on a part of the insole, a near infrared spectroscopy sensor is associated with the body for detecting oxygen saturation in a desired tissue, a controller receiving signal from the sensor and the near infrared spectroscopy sensor, wherein the controller comprises a processor and non-transient memory storing instructions that when executed by the processor causes the controller to send out an alert signal when the sensor measures pressure or weight exceeding a pressure threshold or the near infrared spectroscopy sensor detects an indicator of an evolving deep vein thrombosis (DVT).

In some preferred embodiments of the present disclosure the non-transitory computer-readable medium further contains instructions that cause the processor to perform and analyze the data received from the near infrared spectroscopy sensor to determine a probability for formation of a DVT and adjust the pressure threshold of the postop shoe based on the probability for formation of a DVT.

It will be appreciated by those skilled in the art that the controller may have a main processor or processing unit and a non-transient memory or could be implemented using circuitry.

It will be appreciated by those skilled in the art that the shoe may have a power source that could be placed inside the shoe or could be connected to an external power source.

In some preferred embodiments, the shoe may further include a user interface screen for receiving the alert signal from the controller, receiving instructions from a user, showing charge status of the shoe, showing connection to other systems, turning the central processing unit on or off or for otherwise receiving instructions or conveying messages to the patient.

In one preferred example, the shoe may further have a transceiver for communicating with a remote device to send the alert signal and receive instructions from the patient or a medical professional.

In some preferred embodiments of the present disclosure, the shoe may further comprise a power generator system. The power generator system may include one or more of a piezoelectric, flexible solar panels, and a pressure based power generator.

It will be appreciated by those skilled in the art that one or multiple sensors may be implanted in the body of the shoe, and the sensors could be any type of pressure or other sensor including but not limited to force sensitive resistor (“FSR”) sensors, piezoelectric sensors, strain gauge sensors or other sensors.

In some preferred embodiments, the user interface screen may specify the area of the insole on which the weight has exceeded the predefine threshold.

In some preferred embodiments, the near infrared spectroscopy sensor detecting the indicator of an evolving DVT comprises near infrared spectroscopy detecting an oxygen saturation level passing an oxygen threshold level.

In some preferred examples, the pressure threshold and the oxygen threshold level are calibrated with an initial dataset or a healthy limb. In some other preferred examples, the thresholds may be set based on a predetermined chart.

In another broad aspect, the present disclosure provides a shoe that may comprise a body for receiving part of a patient's foot having an insole area, a sensor placed within the body to measure pressure applied on a part of the insole area of the shoe, a near infrared spectroscopy sensor for connecting to a part the patient's body, a controller receiving signals from the sensor and the near infrared spectroscopy sensor, wherein the controller sends out a signal when the sensor measures pressure or weight exceeding a pressure threshold or the near infrared spectroscopy sensor detects an indicator of an evolving Deep Vein Thrombosis.

In another aspect, a preferred embodiment of the invention is directed to a postop shoe system for detecting deep vein thrombosis (“DVT”) of a patient having an injured limb and a healthy limb. The postop shoe system includes a postop shoe having a sole, the NIRS sensor mounted to the postop shoe such that the NIRS sensor faces skin of the patient in a working configuration and a central processor in communication with the NIRS sensor. The postop shoe is configured for the patient to wear on a foot of the injured limb. The central processor includes a dataset module storing a clinical dataset of oxygen saturation. The central processor is configured to receive oxygen concentration data from the NIRS sensor and compare the received oxygen concentration data to the clinical dataset of oxygen saturation and diagnose an indication of DVT or an absence of DVT.

In a further aspect, a preferred embodiment of the present invention is directed to a postop shoe for monitoring an injured limb of a patient. The postop shoe includes a sole, a pressure sensor mounted to the sole, a NIRS sensor mounted to the sole and a central processor mounted to the shoe. The central processor includes a dataset module storing a clinical dataset of oxygen saturation and a predetermined weight. The central processor is in communication with the pressure sensor to receive weight data. The central processor is also in communication with the NIRS sensor to receive oxygen concentration data. The central processor is configured to compare the weight data to the predetermined weight and the oxygen concentration data to the clinical dataset of oxygen saturation.

### BRIEF DESCRIPTION OF THE SEVERAL VIEWS OF THE DRAWINGS

The foregoing summary, as well as the following detailed description of preferred embodiments of the shoe, system and method of the present application, will be better understood when read in conjunction with the appended drawings. For the purposes of illustrating the system and method for weight loading feedback and DVT prevention, there are shown in the drawings preferred embodiments. It should be understood, however, that the application is not limited to the precise arrangements and instrumentalities shown. In the drawings:

FIG. 1 shows a top perspective view of a smart postop shoe with parts embedded in an insole, in accordance with a first preferred embodiment of the present invention;

FIG. 2 shows a top perspective view of an insole of the smart postop shoe of FIG. 1, wherein sensors are embedded in the insole of the shoe;

FIG. 3 shows a top perspective view of an alternative insole of a preferred smart postop shoe including power generators, batteries, and a power management circuit board in accordance with a preferred embodiment of the present invention;

FIG. 4 is flowchart showing the data flow between different sections of a postop shoe system in accordance with a preferred embodiment of the present invention;

FIG. 5 is a schematic view of a user interface of the smart postop shoe on a computer or a smartphone in accordance with a preferred embodiment of the present invention;

FIG. 6 shows a flowchart of a preferred workflow of the smart postop shoe of FIG. 1;

FIG. 7 is a flowchart showing a Near Infrared Spectroscopy (NIRS) unit working in accordance with a preferred embodiment of the present invention;

FIG. 8 is a flowchart of adapting a new weight-bearing plan based on NIRS sensor and pressure data from preferred sensors of the present invention;

FIG. 9 is a chart example of a weightbearing plan adjustment based on NIRS sensor data in accordance with a preferred embodiment of the present invention; and

FIG. 10 is an example chart showing a correlation between a limb position and muscle oxygenation in accordance with a preferred embodiment of the present invention.

### DETAILED DESCRIPTION OF THE INVENTION

Certain terminology is used in the following description for convenience only and is not limiting. Unless specifically set forth herein, the terms “a”, “an” and “the” are not limited to one element but instead should be read as meaning “at least one.” The words “right”, “left”, “lower” and “upper” designate directions in the drawings to which reference is made. The words “inwardly” or “distally” and “outwardly” or “proximally” refer to directions toward and away from, respectively, the patient's body, or the geometric center of the preferred shoe, system and related parts thereof. The words, “anterior”, “posterior”, “superior,” “inferior”, “lateral” and related words and/or phrases designate preferred positions, directions and/or orientations in the human body to which reference is made and are not meant to be limiting. The terminology includes the above-listed words, derivatives thereof and words of similar import.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Reference is made throughout this disclosure to “one embodiment,” “an embodiment,” “a preferred embodiment” or similar language and means that a particular feature, structure, or characteristic described in connection with the embodiment is included in at least one embodiment of the preferred invention. Thus, appearances of the phrases “in one embodiment,” “in an embodiment,” “in a preferred embodiment” and similar language throughout this disclosure may, but do not necessarily, all refer to the same embodiment.

Moreover, the described features, structures, or characteristics of the preferred invention may be combined in any suitable manner in one or more embodiments. It will be apparent to those skilled in the art that various modifications and variations can be made to the present preferred invention without departing from the scope of the invention. Thus, it is intended that the present preferred invention cover the modifications and variations of this invention provided they come within the scope of the appended claims and their equivalents. Reference will now be made in detail to the preferred embodiments of the preferred inventions.

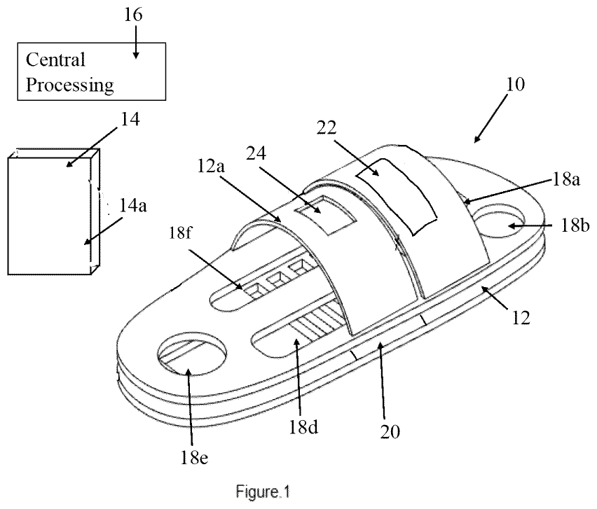

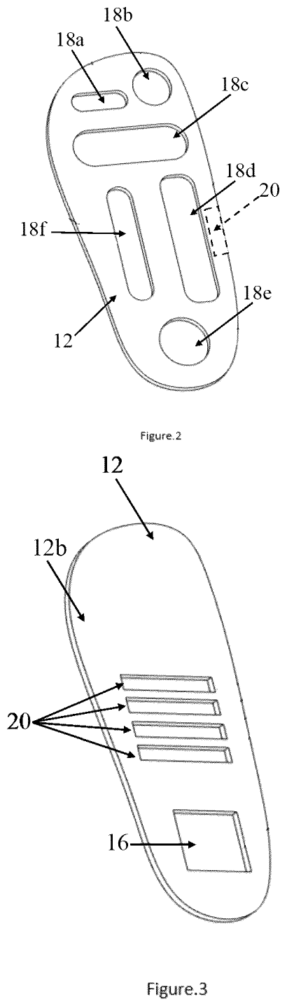

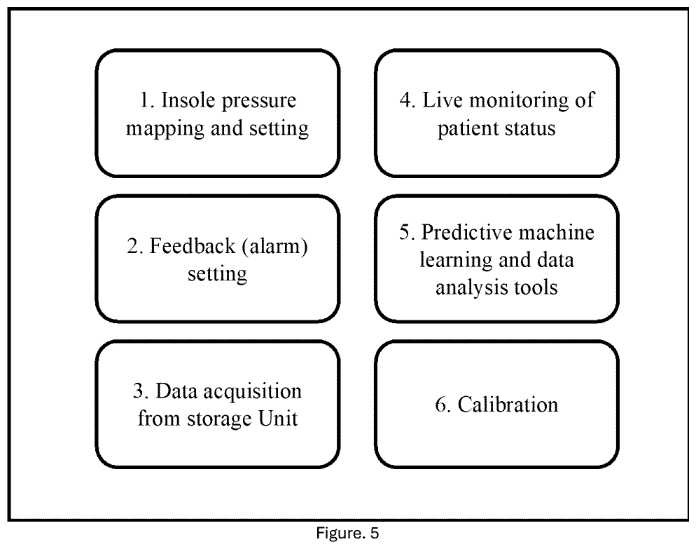

Referring to FIGS. 1-10, Deep Vein Thrombosis (DVT) is a condition that occurs when a blood clot forms in a patient's deep vein in the lower limbs. This clot can form emboli which can block the proper blood flow to different organs including the brain or lungs resulting in a life-threatening event. Weight bearing is a preventive measure against blood clot formation. In a non-weight bearing status following a fracture or surgery, preventive measures include blood-thinner medication, and pneumatic compression therapy.

Early weight bearing following surgery or other treatment is preferred, however, early weight bearing is also preferably weighed against fixation failure. Appropriate weight bearing post-surgery may necessitate programmed weight bearing based on limb circulation to optimize the allowed weight on the affected limb. In a pre-DVT status, it is recommended to bear weight as much as allowed to improve blood circulation and prevent blood clot formation. However, if a DVT forms, weight bearing is preferably restricted to avoid emboli thrown to the lungs which is a life threatening effect.

Limb elevation is commonly advocated for the conservative management of soft tissue edema in several instances, including post-foot and ankle surgery, impending compartment syndrome, flap reconstruction and other similar procedure and conditions. Elevation has been implemented using a standard-size pillow, two pillows or an external fixator. Conventionally, one or two pillows are recommended to elevate the leg above the heart level to decrease swelling. Still, no scientific analysis shows how much elevation and how many pillows (height) will restore the optimum muscle oxygenation during recovery. There is scarce data regarding the “optimum level of limb elevation” and “elevation schedule” after surgery to progressively increase limb dependency to simultaneously allow soft tissue healing and mobilization.

However, the exact elevation height, elevation duration and changes in elevation over time have remained vague and do not provide a clear definition, treatment plan or a limb dependency schedule to train the limb perfusion.

In addition, in many medical applications, we need to have feedback and control over the quantitative external force applied to the lower limbs. This feedback is especially important for fracture care and orthopedic postoperative care to control the amount of weight that the lower limb bears when the patient starts and/or increases mobility.

The optimum oxygenation is speculated to correlate with wound healing, restoring muscle function, and perceived pain. Oxygen saturation (SO2) has been used to monitor free flap training during intermittent elevation and crutch walking. NIRS (near-infrared spectroscopy) devices or sensors provide real-time, accurate and cost-effective SO2 results or data during leg elevation and weight bearing.

In addition, in many medical applications, we need to have feedback and control over the quantitative external force applied to the lower limbs. This feedback is especially important for fracture care and orthopedic postoperative care to control the amount of weight that the lower limb bears.

Studies show that the NIRS data is effective in diagnosing and predicting an evolving DVT. This system may include a transmitter and receiver between seven hundred and eight hundred nanometers (700-800 nm) wavelength. In this system, the NIRS sensor or device can detect the concentration of oxy- and deoxyhemoglobin in the tissue (Total Hb=HbO2+deoxy-Hb). The major difference of this system to the current pulse oximetry is that the NIRS sensor does not require pulsatile blood volume to calculate the parameters having clinic value. Moreover, this system detects hemoglobin located mainly within micro-circulation passages, e.g., capillaries, arterioles, and venules.

NIRS is a noninvasive, safe, and reliable method in detecting oxygen saturated hemoglobin (HbO2) in blood circulation, which has recently shown high sensitivity of approximately ninety-seven percent (97%) for the diagnosis of deep vein thrombosis. NIRS is preferably operator independent. It is also safe to be used for a relatively long time. A study has shown the mean hyperbaric oxygen (“HbO2”) detected by the NIRS sensor. The mean HbO2 values were eighty seven percent, plus or minus six percent (87%±6%) for healthy volunteers, eighty-three percent, plus or minus ten percent (83%±10%) for non-shock patients, eighty-three percent, plus or minus ten percent (83%±10%) for mild shock patients, eighty percent, plus or minus twelve percent (80%±12%) for moderate shock patients and forty-five percent, plus or minus twenty-six percent (45%±26%) for severe shock patients.

Studies show that NIRS is effective in diagnosing and predicting the evolving DVT. This system may include a transmitter and receiver having a wavelength between seven hundred and eighty hundred nanometers (700-800 nm). In this preferred system, the NIRS sensor can detect the approximate concentration of hyperbaric oxygen and deoxyhemoglobin in the tissue (Total Hb=HbO2+Hb). The major difference of this system to the current pulse oximetry detection, which is generally considered the current standard of care, is that the NIRS sensor does not require pulsatile blood volume to calculate the parameters having clinical value. Moreover, this system detects hemoglobin located mainly within microcirculation passages, e.g., capillaries, arterioles, and venules.

In addition, in many medical applications, a medical professional prefers to have feedback and control over the quantitative external forces applied to the lower limbs of the patient. This feedback is especially preferred for fracture care and orthopedic postoperative care to control the amount of weight applied to the lower limb that has been treated.

One way to traditionally educate the patients regarding the appropriate amount of allowed weight-bearing is using a bathroom scale. For example, a plan for a scheduled increase in weight bearing is twenty-five percent (25%) for the first two weeks, followed by fifty percent (50%), seventy-five percent (75%), and then one hundred percent (100%) for each following two weeks. Patients may use a bathroom scale to put pressure and weight until the scale reaches twenty-five percent (25%) of their actual weight. By repeatedly stepping on and off the plate, the patient gains a physical feel for how much weight is twenty-five percent (25%) of the body weight and tries to continue this amount of pressure or force for the next two weeks with crutches assistance or other weight bearing assistance to bear the remainder of their body weight. This is a cumbersome, unpredictable and inaccurate process and is prone to human error.

Patients may also be given certain types of postop shoes to protect a vulnerable part of the patient's sole from external forces or pressure.

For postoperative care, the orthopedic expert may recommend that the patient apply a minimum weight on the affected foot initially and gradually increases the amount of weight based on improvements and their progress in healing.

In sports like soccer, athletes are given specialized shoes, such as spikes, sneakers or athletic shoes, that can measure motion, pressure, and other technical data so that their coaches can analyze the player's performance. These specialized spikes, sneakers or shoes are equipped with several sensors embedded inside a flexible insole and a wireless transmitter sends the player's data to the coach's smartphone, tablet, etc.

More generally, smart postop shoes can provide analytical data such as location, speed, acceleration, DVT prediction and diagnosis, pressure, etc., for various purposes. One of the main issues of this type of footwear is charge consumption and the amount of time it takes to recharge the batteries. Meanwhile, the user is unable to wear the shoes while the battery is recharging.

Referring to FIGS. 1 and 2, a postop shoe or shoe system, generally designated 10, is preferably utilized for detecting deep vein thrombosis (“DVT”) of a patient having an injured limb and a healthy limb. The present disclosure describes the smart postop shoe 10 that assists the patient with understanding weight-bearing on a specific point or areas of a sole 12 of the shoe 10 and with awareness of how much force or weight is being applied to the treated limb during recovery. Simultaneously, if the exerted weight exceeds the preset or predetermined value or weight, the postop shoe 10 may send an alarm employing textual, vibrational, audible and/or visual signal to the patient. The warning or indicator may be a textual message sent to the patient's smartphone 14, a combination vibration and textual message sent to the patient's smartphone 14, a beeping sound emanating from the patient's smartphone 14, visual lights and beeping sounds from the smartphone 14 or a message sent to a device provided to the patient by the healthcare provider. The specific alerts or indicators warn the patient quickly or instantly if the weight exceeds the scheduled percentage of weight or predetermined weight. The messages may be depicted on a display 14a of the smartphone 14 or otherwise communicated to the patient.

In some preferred embodiments, the system of the smart postop shoe 10 may set the weightbearing percentage after casting or after the surgery using the software connected to a central processing unit or a central processor 16 of the smart postop shoe 10 utilizing wireless communication. The central processor 16 may be comprised of an application associated with the smartphone 14, may be remote and in communication with the postop shoe 10 and the smartphone 14, may be mounted directly to the postop shoe 10 or may be comprised of a combination of software and hardware directly mounted to the postop shoe 10 and/or the smartphone 14 or remote from the postop shoe 10 and the smartphone 14.

The postop shoe 10 of the preferred embodiment includes sensors 18 mounted thereto to collect data regarding the shoe 10 and patient and transmit the collected data to the central processor 16. The sensors 18 may include weight sensors or load transducers that detect the weight applied by the patient on the postop shoe 10. The pressure or weight data, such as an average pressure and weight detected at each area of the sole 12, collected from the sensors 18 is recorded and stored inside a storage memory unit of the central processor 16 for further analysis. Moreover, an allowed weight bearing percentage or predetermined weight or force, which is preferably stored in the central processor 16, is coupled with or compared to the data received from the sensors 18, such as a NIRS sensor, which is constantly checking or collecting data regarding the normal, pre-DVT and evolving DVT status. The allowed percentage, predetermined percentage or predetermined weight or force is also adjusted based on the DVT status to avoid exceeding weight bearing in an evolving DVT status.

In the first preferred embodiment, six (6) sensors or sensor plates 18 are placed on or mounted within different areas of the sole 12 to differentiate each region's weight bearing. The sensors 18 may include a first sensor 18a, a second sensor 18b, a third sensor 18c, a fourth sensor 18d, a fifth sensor 18e and a sixth sensor 18f. The first, second, third, fourth, fifth and sixth sensors 18a, 18b, 18c, 18d, 18c, 18f are preferably comprised of pressure sensors, load cells or load transducers but are not so limited and may be comprised of alternative sensors that collect data regarding the patient and/or the conditions of or adjacent to the shoe 10, such as NIRS sensors, temperature sensors, pulse oximeters, humidity sensors or related sensors for collecting data and transmitting the data to the central processor 16. The sensors 18 preferably sense the force, pressure and/or weight applied to the associated area of the sole 12, but the sensors 18 may also produce electric charges to be stored in rechargeable batteries 20 that are mounted in or on the postop shoes 10. However, the sensors 18 may not be the only charge producers on the smart postop shoe 10. Flexible solar panels 22 may also be placed on an upper side of the shoe 10 to produce enough electric charge to keep the processing unit or central processor 16 up and running at times when the rechargeable batteries 20 run out or for recharging, and no power supply is available.

Referring to FIG. 1, a smart screen 24 may be positioned on the upper side of the shoe 10 or on an upper strap 10a of the shoe 10 to provide a good interface for the user and indicate battery charge status, provide an on and off switch, and/or connection to the smartphone 14, central processor 16 or computer. The smart screen 24 may be connected to the central processing unit or central processor 16, which may be embedded in or on the sole 12. The rechargeable batteries 20 may also be comprised of several relatively flat batteries 20 that are mounted on or in the sole 12.

In some preferred embodiments, a charge produced by the solar panels 22 and piezoelectric power generators that may be associated with the sensors 18 recharges the batteries 20. In some preferred embodiments, the postop shoe 10 may also transfer the data of the charge of the batteries 20 to the central processing unit or the central processor 16 and the charge level may be displayed on the smart screen 24 or the display 14a of the smartphone 14.

In some preferred embodiments, the healthcare provider may have access to the pressure data stored in the central processor 16 by connecting the postop shoe 10 to software and a graphical user interface (“GUI”) may be used to assist the healthcare provider in reviewing, manipulating and interpreting the pressure data. For example, the GUI may assist the healthcare provider with conducting further analysis on the data and creating related charts. The data that is recorded, collected and acquired by the sensors 18 is preferably stored in the data storage unit of the central processor 16. The software of the central processor 16 may also be used to calibrate the shoe 10, set and produce alarm setting and determine a weight-bearing percentage or predetermined weight target for each of the regions on the sole 12.

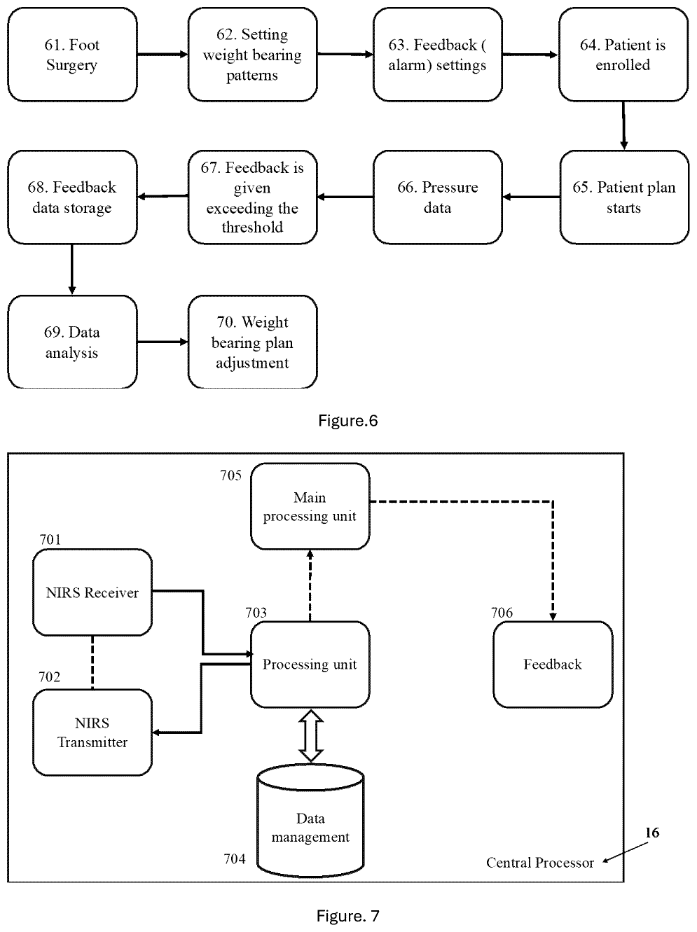

Referring to FIG. 6, a workflow for using the postop shoe 10 may commence after a patient has a foot surgery 61. The patient preferably starts using the shoe 10 by wearing the shoe 10 on the foot where surgery was performed. The patient initially becomes mobile with the aid of crutches or other assistive devices and the patient applies only a percentage of their weight on the sole 12 of the postop shoe 10, which is a setting weight bearing patterns step 62. This stage of care could take two weeks but is not so limited and may extend longer or be comprised of a shorter timeframe based on the patient, surgery or other factors. Based on the type of injury and surgery, the provider preferably decides the allowed weight bearing percentage or a predetermined weight and the incremental increase of the predetermined weight over the time that the patient is anticipated to be in recovery from the surgery, which is an alarms setting step 63. For instance, if the patient exceeds the predetermined pressure or weight above twenty-five percent (25%) of the patient's total weight pressure, an alerts (vibration or noise) may start ringing and vibrating or a textual warning on the display 14a or smart screen 24 may be represented or sent to the patient. The data regarding weight or force applied to the sole 12 by the patient during use is preferably conveyed or displayed to the patient in this step such that the patient gains awareness of or a feel for the weight being applied to the foot, which comprises a patient is trained step 64. The patient starts using 65 the system and pressure data 66 is collected by the sensors 18 and transmitted to the central processor 16. Alarms will turn on for excessive pressure 67, such as when data collected from the sensors 18 exceeds the predetermined weight, which is preferably recognized by the central processor 16. The alarm data is stored 68 in the central processor 16 and the data is preferably analyzed 69 by the central processor 16. In addition to providing a warning or alarm to the patient, the central processor 16 may notify the healthcare provider that the weight on the shoe 10 has been exceeded by the patient. After exerting a weight percentage, as captured or acquired by the sensors 18, e.g., twenty-five percent (25%) of body weight, for a certain period, e.g., two weeks, the provider may allow the patient to gradually increase the percentage of weight bearing by setting the new alert thresholds or by increasing the predetermined weight on the sole 12 of the postop shoe 10, which is a weight bearing increases if needed step 70. As a non-limiting example, the predetermined weight may increase from twenty-five percent (25%) of the patient's weight to fifty percent (50%) of body weight for the next two weeks using the graphical user interface. Depending on the progress in healing, bone quality, type of fracture, type of surgery, quality of fixation, and provider's preference, an increase in the weight bearing to one hundred percent (100%) may be scheduled.

The allowed weight bearing or predetermined weight can be adjusted for each of the three main areas of the foot including hindfoot, where the fifth sensor 18e is positioned, midfoot, where the fourth and sixth sensors 18d, 18f are positioned, and forefoot, wherein the first, second and third sensors 18a, 18b, 18c are located. For instance, in a bunion surgery in the forefoot region, the patient might be allowed to apply full weight bearing in the heel (hindfoot), and partial weight bearing in the midfoot and no weight bearing in the forefoot by setting a predetermined hindfoot weight of one hundred percent (100%), a predetermined midfoot weight between zero and one hundred percent (0%-100%) and a predetermined forefoot weight of zero percent (0%), which is preferably stored in the central processor 16. A threshold or stored predetermined weight or force can be adjusted separately for each region of the foot and is preferably stored in the central processor 16. In the preferred embodiment, the first, second and third sensors 18a, 18b, 18c may be comprised of a forefoot sensor 18a, 18b, 18c, the fourth and sixth sensors 18d, 18f may be comprised of a midfoot sensor 18d, 18f and the fifth sensor 18e may be comprised of a hindfoot sensor 18e. The forefoot sensor 18a, 18b, 18c is preferably mounted to a forefoot portion of the sole 12, the midfoot sensor 18d, 18f is preferably mounted to a midfoot portion of the sole 12 and the hindfoot sensor 18e is preferably mounted to a hindfoot portion of the sole 12. The forefoot, midfoot and hindfoot sensors 18a, 18b, 18c, 18d, 18e, 18f are preferably comprised of pressure sensors, but are not so limited and may be comprised of other sensors, such as the NIRS sensor 801, 804 or other sensors. The first, second, third, fourth, fifth and sixths sensors 18a, 18b, 18c, 18d, 18c, 18f may also be otherwise arranged on or near the sole 12 of the shoe 10 compared to the positioning and configuration of the first, second, third, fourth, fifth and sixths sensors 18a, 18b, 18c, 18d, 18e, 18f shown in FIG. 2. For example, the forefoot, midfoot and hindfoot sensors (now shown) may be comprised of single sensors or the shoe 12 may not include each of the forefoot, midfoot and hindfoot sensors 18a, 18b, 18c, 18d, 18c, 18f without significantly impacting the function and operation of the preferred shoe system 10.

The postop shoe 10 may alternatively include outsoles (not shown) conforming to unload areas of the foot. For instance, a rocker bottom outsole can be attached to the postop shoe 10 to allow loading of the midfoot while unloading the hindfoot and forefoot. The rocker bottom outsole is especially helpful in patients with a leg cast or boot which cause limited ankle motion. In this situation, a threshold or predetermined weight is adjusted accordingly such that the alarm is produced based on the data from the sensors 18 when desired by the healthcare provider.

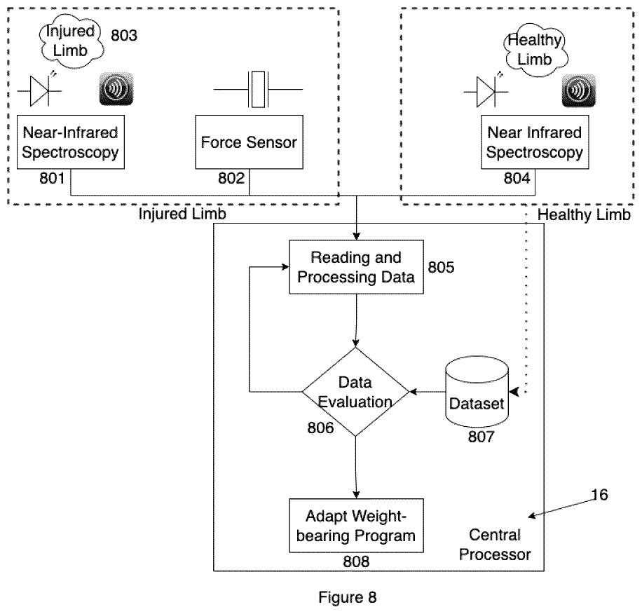

Referring to FIG. 7, a general configuration for a near infrared spectroscopy (“NIRS”) module in the central processor 16 and a workflow for NIRS includes a NIRS transmitter 702, a NIRS receiver 701, a processing unit 703, a data management unit 704, a main processing unit 705 and an alert module 706. The NIRS transmitter 702 is the transmitter of the near infrared wavelength. The processing unit 703 may send signals to activate the NIRS transmitter 702. The NIRS transmitter 702 may send light signals into the patient's tissue, which may include three wavelengths traveling a predetermined distance. The attenuation of the light signal may be measured based on Lambert's Law and include first, second and third wavelength intensity. This attenuation is designed to produce a representation of the blood oxygen saturation level within different subject tissue attenuation. The NIRS receiver 701 is a sensor that detects the wavelengths transmitted through the subject tissue to determine the blood oxygen at a determined distance. The oxygen level and sensor calibration are processed at the processing unit 703. The attenuation from Lambert's law can determine the change in chromophore concentration. Based on the pre dataset from the NIRS attenuation of various tissues and the patient's healthy tissue, the DVT can be predicted or diagnosed. The prediction or diagnose of DVT is based on the healthy limb prior data or the clinical dataset of oxygen saturation for various limbs. It has been shown that the thrombosis leg has a ratio (Δ[HBO2]Δ[HB]) of around ten (10), where the healthy leg has this ratio above eleven (11). This level difference and any drop in the ratio indicates that the DVT can be predicted or diagnosed. Prior to measuring data, a soft tissue calibration as the reference point is used to detect any artifacts or noises in the NIRS receiver 701 or the NIRS transmitter 702. The dataset “DVTipd” is a hypothetical dataset with five hundred (500) subjects suspected of having DVT that can be used as the prior dataset as a method to compare the oxygen saturation level in the patient leg with the prior clinical DVT dataset. The DVT status is transmitted to the main processing unit 705 (evaluating with healthy limb or prior dataset, such as in a data evaluation step 806 of FIG. 8) to adapt the new weight-bearing plan. The new weight-bearing plan can increase the predetermined pressure or weight in attempts to prevent the DVT or inform the patient's medical provider if DVT is diagnosed or suspected. The processing unit 705 may have additional functionality, such as was explained above and shown in FIG. 1. If the main processing unit 703 detects any DVT occurrence or predicts a DVT the main processing unit 705 preferably notifies or sends a message to the patient and the physician, such as to the smart screen 24, the display 14a of the smartphone 14 or to another system or device that may be accessed by the patient or the physician. The sensors 18 may be utilized to measure force for transmission to the central processor 16, which is used to measure the weight-bearing on predefined spots of the sole 12 by the patient's feet.

Referring to FIGS. 1-8, the prediction of DVT and customized weight bearing may be planned by the initial clinical dataset and based on pre-surgical or concurrent monitoring of a patient's healthy limb. The data may be stored in a dataset 807 and analyzed in a data evaluation module or step 806. Data may be collected and recorded at a patient's healthy limb by a healthy limb NIRS sensor 804. The healthy limb data may be utilized to tune the parameters of a weight-bearing plan 808 for the specific patient. Moreover, the data collected from the patient's healthy limb may be used as the oxygen saturation level for predicting the DVT in the patient's injured limb during healing process. FIG. 8 provides an explanation of a decision flowchart for adapting the new weight-bearing program according to data collected from the patient's healthy and injured limbs, wherein data from an injured limb NIRS sensor 801, an injured limb pressure or force sensor 802 and the healthy limb NIRS sensor 804 are transmitted to a reading and processing data module 805 of the central processor 16 or the dataset 807, the data is evaluated in a data evaluation module 806 and the evaluation drives adaptation of the weight bearing program for the patient based on an adapt weight-bearing program module 808.

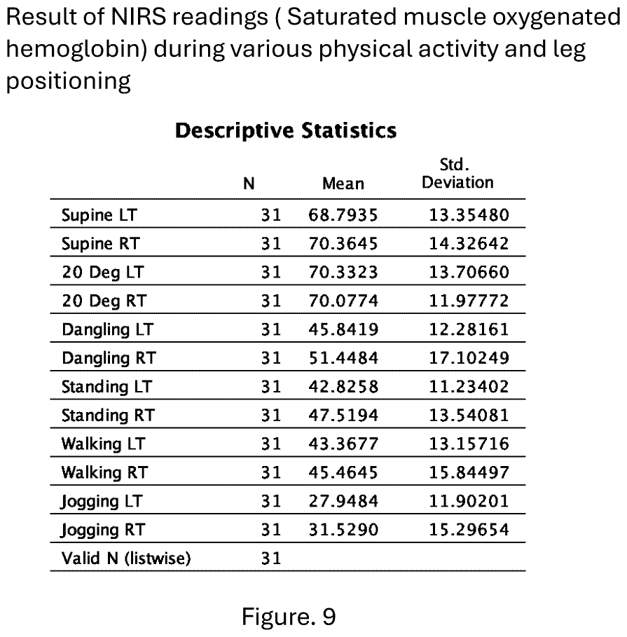

Referring to FIGS. 8 and 9, the allowed weight bearing for the patient may be adjusted based on the continuous reading of the HbO2 by the NIRS sensors 801, 802. For example, if the patient is allowed to put up to twenty-five percent (25%) of his/her total weight on the affected or injured limb, this plan is followed as long as the injured limb NIRS sensor 801 shows that the HbO2 reading falls within the normal range or does not fall below a predetermined HbO2 value. When the data collected from the injured limb NIRS sensor 802 shows that HbO2 values fall below the normal range or below the predetermined HbO2 value, which is sensitive for a developed DVT, the patient and/or healthcare provider is alarmed and advised that weightbearing should be hampered until the condition of the injured limb or the weight bearing plan can be evaluated.

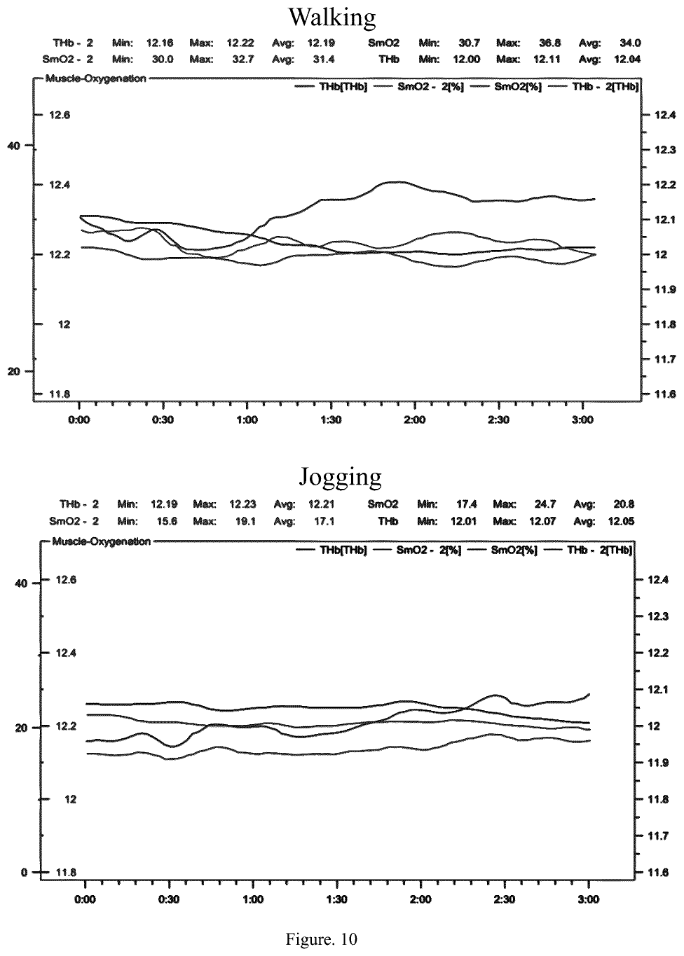

Referring to FIG. 10, limb position and elevation may be adjusted, or the patient may be instructed to adjust limb position and elevation and the central processor 16 may be programmed to optimize the oxygenation based on the optimum oxygen level and adjust saturated oxygen pressure (SPO2) and optimize fluctuations in SPO2.

It would be appreciated by those skilled in the art that other methods can be alternatively used to implement the NIRS readings into a smart and interactive weight bearing program such as using a predefined schedule, instructions received by the device from the surgeon or physician etc. In one preferred example, the post-op shoe 10 may be able to connect to the central processor 16, which may be comprised of a server (via internet or other known method), to communicate the data to the server for the purpose of further monitoring the improvement of the patient by the health care professional. In a preferred example, it can also receive information from the server to adjust the weight bearing schedule.

In the preferred present invention, the smart sole 12 and postop shoe 10 assist the patient in their awareness of the percentage of weight-bearing on a specific point of the foot including rear, middle and front of the foot as well as inside and outside of the foot. Simultaneously, if the exerted weight exceeds the pre-set or predetermined weight, system of the postop shoe 10 preferably sends an alarm employing vibration and audiovisual warnings or alarms to the patient, such as by using light and beep sounds. The specific alerts warn the patient quickly, nearly instantly, if the detected weight exceeded the scheduled percentage of weight.

The provider or designer can set the weight-bearing percentage for management of a diabetic foot ulcer, after casting, or after the surgery using the software and related modules of the central processing unit or processor 16 of the smart postop shoe system utilizing wireless communication. Furthermore, the data from the sensors 18, which are preferably comprised of pressure sensors 18, 18a, 18b, 18c, 18d, 18c, 18f (average pressure and weight of each area of the sole 12) is recorded and stored inside the storage memory unit or data management for further analysis. Moreover, the allowed weight bearing percentage or predetermined weight is coupled with the data received from the NIRS sensors 801 which is constantly checking the normal, pre-DVT, and evolving DVT status. Allowed percentage or predetermined weight is also adjusted based on the DVT status to avoid exceeding weight bearing in an evolving DVT status.

The NIRS data measuring muscle oxygenation is set to warn the patient about a continued drop in oxygen level requiring a change in leg position and the intensity and duration of weight bearing. The affected or injured lower limb has impaired venous return resulting in swelling following and extended period of weight bearing and leg hanging. Real time measurement of the oxygen drop can prevent hypoxemia of the tissues to prevent wound healing issues, swelling, and pain.

Referring to FIGS. 1-3, six (6) sensors or sensor plates 18 may be placed on different areas of the sole 12 to differentiate weight-bearing on different parts of the foot, including forefoot, midfoot and hindfoot. The sensors 18 measure the pressure and weight applied to the specific area of the sole 12 by the injured limb or the healthy limb. The six sensors 18 are not limiting and the system may operate without significant impact with less or more sensors 18, such as a single sensor 18, three sensors 18 in the three described areas of the foot or more sensors 18. A wireless or cable charger may be connected to charge the battery 20 placed in the sole 12. The smart screen 24 may be placed on an upper side 12a of the shoe 12 to provide a good interface for the user and indicate battery charge status, an on and off switch, connection to the smartphone or computer status and other features. The smart screen 24 is preferably connected to the central processing unit or central processor 16, which may be placed on a lower portion 12b of the sole 12 with several flat batteries 20. The sole 12 is connected to the smartphone 14, which may include smart devices such as a phone, watch or any operating system. The smartphone 14 preferably has wireless capability, such as Bluetooth, and wireless in real-time or offline to download and analyze the collected or acquired data.

The provider is preferably able to access the pressure data by connecting the postop shoe 10 to the software by wired or wireless systems, the GUI may conduct further analysis on data and may draw related charts. The data collected, acquired and recorded by the sensors 18 is preferably stored in the data storage unit or data management unit 704 of the central processor 16. The central processor 16 is also preferably used to calibrate the shoe 10, the alarm settings and to determine the weight-bearing percentage or a predetermined weight for the insole 12, preferably for each of the regions on the sole 12.

Referring to FIG. 6, in the workflow for using the postop shoe 10, the patient starts using the shoe. With the aid of crutches or other assistive devices, the patient applies only a percentage of their weight on the postop sole 12. This stage of care could take two weeks. Based on the type of injury and surgery, the provider may decide the allowed weight bearing percentage or predetermined weight and the incremental increase over the time. For instance, if the patient exceeds the pressure above twenty-five percent (25%) of the total weight pressure, the alerts (vibration, textual or audiovisual) will send a message and/or start ringing and vibrating. After exerting a weight percentage, e.g., twenty-five percent (25%) of body weight, for a certain period, e.g., two weeks, the provider may allow the patient to gradually increase the percentage of weight-bearing or predetermined weight by setting the new alert thresholds on the sole 12, e.g., fifty percent (50%) of body weight for the next two weeks using the GUI. Depending on the progress in healing, bone quality, type of fracture, type of surgery, quality of fixation, and provider's preference, an increase in the weight-bearing to one hundred percent (100%) is scheduled.

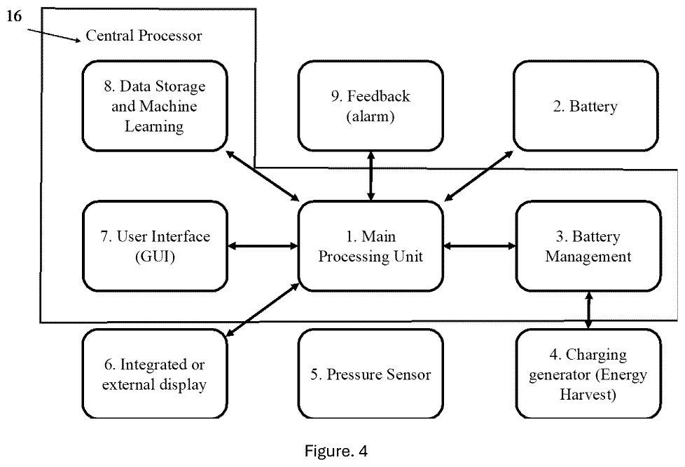

Referring to FIG. 7, the general configuration for the NIRS module in the central processor 16 and a workflow for the NIRS system includes the NIRS transmitter 702 of the near infrared wavelength and the processing unit 703 that may send a signal to the NIRS transmitter 702 to activate the NIRS transmitter 702. The NIRS transmitter 702 may send light signals into the patient's tissue, including three wavelengths traveling for a predetermined distance. The attenuation of the light signal is measured within Lambert's Law and have first, second, and third wavelength intensity. This attenuation shows the blood oxygen saturation level within differences in the patient's tissue attenuation. The NIRS receiver 701, which is a sensor, detects the wavelengths transmitted through the patient's tissue to determine the blood oxygen at the determined distance d. The oxygen level and sensor calibration are processed at processing unit 703. The attenuation from Lambert's Law can determine the change in chromophore concentration. Based on the pre dataset from the NIRS attenuation of various tissues in the data management unit 704 and the subject healthy tissue, the DVT can be predicted or diagnosed. The same data regarding muscle oxygenation level drop may be generated and May be transmitted. The DVT status is transmitted to the main processing unit 705 to adapt, develop or suggest a new weight-bearing plan. The new weight-bearing plan can increase the pressure or predetermined weight to treat and/or prevent the DVT or inform the patient's healthcare provider if DVT is diagnosed. The other functions of the main processing unit 705 were explained herein, particularly with respect to FIG. 4. If the main processing unit 705 detects any DVT occurrence or predicts a DVT, the central processor 16 notifies the patient, the healthcare provider and the physician with the alert module 706.

Referring to FIGS. 1-10, The postop shoe 10 has the sole 12 and is configured for the patient to wear on the foot of the injured limb. The postop shoe system 10 includes the postop shoe 10 having the sole 12. The NIRS sensor 801 is mounted to the postop shoe 10 such that the NIRS sensor 801 faces skin of the patient in a working configuration. The NIRS sensor 801 may, for example, be mounted proximate or replace and of the sensors 18, which are typically designed for pressure sensing but may comprise the NIRS sensor 801. The NIRS sensor 801 is preferably mounted to the sole 12 but is not so limited and may be mounted nearly anywhere on the shoe 10 or remove from the shoe 10 to facilitate acquiring the oxygen concentration data in and near the patient's injured limb. The NIRS sensor 801 preferably includes the NIRS transmitter 702 and the NIRS receiver 701, although the NIRS transmitter 702 is preferably mounted to the shoe 10 and the NIRS receiver 701 is preferably in communication with the central processor 16.

The central processor 16 is in communication with the NIRS sensor 801 and including the dataset module 807 storing a clinical dataset of oxygen saturation, preferably among other data and information. The pressure sensor 18 is mounted to the sole 12 and is in communication with the central processor 16. The central processor 16 is configured to receive weight data from the pressure sensor 18 in the working configuration. The dataset module 807 stores a predetermined weight, which may be added, modified and changes for individual patients or based on medical provider preferences or specific patient injuries and levels of recovery. The central processor 16 is configured to compare the predetermined weight to the weight data and determine if a first weight of the weight data exceeds the predetermined weight. The central processor 16 is configured to transmit a weight alarm to the patient when the first weight exceeds the predetermined weight, thereby indicating that the patient is applying excessive weight to the injured limb compared to the recommended weight of the medical professional based on their level of recovery.

The central processor 16 is configured to receive oxygen concentration data from the NIRS sensor 801 and compare the received oxygen concentration data to the clinical dataset of oxygen saturation to diagnose an indication of DVT or an absence of DVT. The weight and DVT data acquisition and comparisons are preferably conducted in parallel while the patient is wearing the shoe 10 to monitor the patient's recovery, monitor the weight that the patient is applying to the injured limb and to monitor potential DVT or provide indications that DVT is unlikely for the patient. The central processor 16 is configured to transmit an alarm to the patient when the indication of DVT is diagnosed based on the comparison of the received oxygen concentration data to the clinical dataset of oxygen saturation. The alarm may be comprised of a message sent to the smartphone 14, a message displayed on the smart screen 24, sounds from the smartphone 14 or smart screen 24 prompted by the central processor 16 or other communication indicating to the patient that a potential DVT is diagnosed based on the comparison.

The central processor 16 includes the dataset module 807 storing a clinical dataset of oxygen saturation and a predetermined weight. The central processor 16 is in communication with the pressure sensor 18 to receive weight data and is in communication with the NIRS sensor 801 to receive oxygen concentration data. The central processor 16 is configured to compare the weight data to the predetermined weight and the oxygen concentration data to the clinical dataset of oxygen saturation. The shoe system 10 also preferably includes the healthy limb NIRS sensor 804 and the central processor 16 is configured to receive healthy limb oxygen concentration data. The clinical dataset of oxygen saturation includes the received healthy limb oxygen concentration data from the healthy limb NIRS sensor 804.

The shoe 10 also includes the smart screen 24 mounted to the upper side of the postop shoe 10. The smart screen 24 is configured to display a battery charge status and alerts from the central processor 16. The shoe 10 may further include the battery 20 mounted to the shoe 10. The battery 20 may be connected to the solar panel 22 and configured to receive electrical charge from the solar panel 22. The battery 20 is preferably in communication with the central processor 16 to power the central processor 16 and transmit data regarding at least a charge level of the battery 20.

The preferred shoe system 10 may also be employed in a preferred method for monitoring a patient's injured limb following surgery or other injury. The shoe system 10 preferably includes the central processor 16 with the dataset module 807 storing a clinical dataset of oxygen saturation including a predetermined normal oxygen saturation, a first predetermined weight and a second predetermined weight. The method includes receiving initial weight data from the pressure sensor 18 at the central processor 16, receiving initial oxygen concentration data from the NIRS sensor 801 at the central processor 16, comparing the weight data to a predetermined weight with the central processor 16, receiving second weight data from the pressure sensor 18 following a predetermined period and comparing the second weight data to the second predetermined weight. The second predetermined weight is greater than the first predetermined weight. The predetermined period may be nearly any timeframe desired by the medical professional, such as several minutes to several hours or ten days to three weeks. In addition, the predetermined period may change over time, such as by acquiring data every several seconds or minutes when the sensor 18 detects an increase in weight or pressure and, in contrast, longer time periods when the sensor 18 detects no or zero weight being applied to the shoe 10.

In the preferred method, the central processor 16 may transmit an alert to the patient or a medical professional when a first weight from the sensor 18 of the weight data exceeds the first predetermined weight during the predetermined period. The preferred method may also compare the initial oxygen concentration data to the clinical dataset of oxygen concentration and transmit an alert when a first oxygen concentration of the initial oxygen concentration data is less than the predetermined normal oxygen saturation. The alert may urge the patient to evaluate the injured limb or may suggest that the patient seek medical attention. The alert may alternatively be transmitted to the medical professional who preferably evaluates the data to determine how to proceed with the patient's treatment.

The first predetermined weight may be twenty to thirty percent of the body weight of the patient and the second predetermined weight may be forty-five to fifty-fiver percent of the body weight. The first and second predetermined weights are not so limited and

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

I/We claim:

1. A postop shoe system for detecting deep vein thrombosis (“DVT”) of a patient having an injured limb and a healthy limb, the postop shoe system comprising:
a postop shoe having a sole, the postop shoe configured for the patient to wear on a foot of the injured limb;
a NIRS sensor mounted to the postop shoe such that the NIRS sensor faces skin of the patient in a working configuration;
a central processor in communication with the NIRS sensor, the central processor including a dataset module storing a clinical dataset of oxygen saturation the central processor configured to receive oxygen concentration data from the NIRS sensor and compare the received oxygen concentration data to the clinical dataset of oxygen saturation and diagnose one of an indication of DVT and an absence of DVT.

2. The postop shoe system of claim 1, wherein the NIRS sensor is mounted to the sole.

3. The postop shoe system of claim 1, wherein the NIRS sensor includes a NIRS transmitter and a NIRS receiver.

4. The postop shoe system of claim 1, wherein the central processor is configured to transmit an alarm to the patient when the indication of DVT is diagnosed based on the comparison of the received oxygen concentration data to the clinical dataset of oxygen saturation.

5. The postop shoe system of claim 1, further comprising:
a forefoot sensor mounted to a forefoot portion of the sole;
a midfoot sensor mounted to a midfoot portion of the sole; and
a hindfoot sensor mounted to a hindfoot portion of the sole, the forefoot, midfoot and hindfoot sensors comprised of pressure sensors.

6. The postop shoe system of claim 5, wherein the forefoot sensor comprises first, second and third sensors, the midfoot sensor comprises fourth and sixth sensors and the hindfoot sensor comprises a fifth sensor.

7. The postop shoe system of claim 1, further comprising:
a pressure sensor mounted to the sole, the pressure sensor in communication with the central processor, the central processor configured to receive weight data from the pressure sensor in the working configuration, the dataset module storing a predetermined weight, the central processor configured to compare the predetermined weight to the weight data and determine if a first weight of the weight data exceeds the predetermined weight.

8. The postop shoe system of claim 7, wherein the central processor is configured to transmit a weight alarm to the patient when the first weight exceeds the predetermined weight.

9. A postop shoe for monitoring an injured limb of a patient, the postop shoe comprising:
a sole;
a pressure sensor mounted to the sole;
a NIRS sensor mounted to the sole;
a central processor mounted to the shoe, the central processor including a dataset module storing a clinical dataset of oxygen saturation and a predetermined weight, the central processor in communication with the pressure sensor to receive weight data and in communication with the NIRS sensor to receive oxygen concentration data, the central processor configured to compare the weight data to the predetermined weight and the oxygen concentration data to the clinical dataset of oxygen saturation.

10. The postop shoe of claim 9, wherein the pressure sensor includes first, second, third, fourth, fifth and sixth sensors.

11. The postop shoe of claim 9, further comprising:
a healthy limb NIRS sensor, the central processor configured to receive healthy limb oxygen concentration data, the clinical dataset of oxygen saturation including the received healthy limb oxygen concentration data.

12. The postop shoe of claim 9, wherein the NIRS sensor includes a NIRS transmitter and a NIRS receiver.

13. The postop shoe of claim 9, further comprising:
a smart screen mounted to an upper side of the postop shoe, the smart screen configured to display a battery charge status and alerts from the central processor.

14. The postop shoe of claim 9, further comprising:
a solar panel mounted to an upper side of the postop shoe; and
a battery mounted to the shoe, the battery connected to the solar panel and configured to receive electrical charge from the solar panel, the battery in communication with the central processor.

15. A method for monitoring a patient's injured limb following surgery or other injury with a postop shoe system having a central processor with a dataset module storing a clinical dataset of oxygen saturation including a predetermined normal oxygen saturation, a first predetermined weight and a second predetermined weight and a postop shoe with a sole, a pressure sensor and a NIRS sensor, the method comprising:
receiving initial weight data from the pressure sensor at the central processor;
receiving initial oxygen concentration data from the NIRS sensor at the central processor;
comparing the weight data to a predetermined weight with the central processor;
receiving second weight data from the pressure sensor following a predetermined period;
comparing the second weight data to the second predetermined weight, the second predetermined weight being greater than the first predetermined weight.

16. The method of claim 15, wherein the predetermined period is ten days to three weeks.

17. The method of claim 15, wherein the first predetermined weight is twenty to thirty percent of a body weight of the patient and the second predetermined weight is forty-five to fifty-five percent of the body weight.

18. The method of claim 15, further comprising:
transmitting an alert when a first weight of the weight data exceeds the first predetermined weight during the predetermined period.

19. The method of claim 15, further comprising:
comparing the initial oxygen concentration data to the clinical dataset of oxygen concentration; and
transmitting an alert when a first oxygen concentration of the initial oxygen concentration data is less than the predetermined normal oxygen saturation.

20. The method of claim 19, wherein the alert urges the patient to elevate the injured limb.
